(19) United States
(12) Patent Application Publication
(10) Pub. No.: US 2006/0095037 A1
(43) Pub. Date: May 4, 2006

(54) Connector assemblies for connecting a bone anchor to a fixation element

(76) Inventors: Bryan S. Jones, Quincy, MA (US); Matthew Hannen, Boston, MA (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 10/977,345

(22) Filed: Oct. 29, 2004

(51) Int. Cl. A61F 2/30

(52) U.S. Cl. 606/61

(57) ABSTRACT

A connector assembly for connecting a bone anchor to a fixation element includes an anchor receiving member, a housing, an intermediate member, a first closure member, and a second closure member. The anchor receiving member has a first portion having an opening for receiving a bone anchor. The housing has a first passage for receiving a fixation element, a second passage for receiving the second portion of the anchor receiving member, and a third passage intersecting the first passage and the second passage. The intermediate member is positionable within the third passage and has a seat for receiving the fixation element and a distal end configured to engage the second portion of the anchor receiving member. The first closure member is positionable within the third passage to engage the intermediate member and the second closure member is positionable within the third passage to engage the fixation element.

### BACKGROUND

Fixation elements may be used in orthopedic surgery to align and/or fix a desired relationship between anatomic structures. In the case of spinal surgery, for example, a spinal fixation element, such as a rod, plate, or cable, may be anchored to one or more vertebrae by attaching the fixation element to one or more bone anchors, such as a hook, a bolt, a wire, or a screw. The spinal fixation element may be contoured to a desired configuration, and once installed, the fixation element may hold the vertebrae in a desired spatial relationship, until, for example, desired healing or spinal fusion has taken place, or for some longer period of time. Connecting a fixation element to conventional bone anchors can be challenging and time consuming as it is often difficult to align the fixation element with each of the bone anchors. Accordingly, there is a need for improved devices and methods for facilitating the connection of a fixation element to one or more bone anchors.

### SUMMARY

Disclosed herein are connector assemblies and methods for connecting a bone anchor to a fixation element. The connector assemblies and methods disclosed herein are particularly suited to connecting a bone anchor anchored in a vertebra to a spinal fixation element, such as a rod, plate, or cable.

In one exemplary embodiment, a connector assembly for connecting a bone anchor to a fixation element comprises an anchor receiving member, a housing, an intermediate member, a first closure member, and a second closure member. In the exemplary embodiment, the anchor receiving member may have a first portion having an opening for receiving at least a portion of a bone anchor and a second portion. The housing, in the exemplary embodiment, may have a first passage for receiving a fixation element, a second passage for receiving the second portion of the anchor receiving member, and a third passage intersecting the first passage and the second passage. The exemplary intermediate member may be positionable within the third passage and may have a seat for receiving the fixation element and a distal end configured to engage the second portion of the anchor receiving member. In the exemplary embodiment, the first closure member may be positionable within the third passage to engage the intermediate member and the second closure member may be positionable within the third passage to engage a fixation element positioned in the seat of the intermediate member.

An exemplary method of connecting a bone anchor with a fixation element comprises positioning a portion of a bone anchor within an opening of an anchor receiving member of a connector assembly, positioning a fixation element within a passage of a housing of the connector assembly, rotating the anchor receiving member relative to a housing, securing the anchor receiving member in a desired orientation relative to the housing, and securing the fixation element relative the housing independently of securing the anchor receiving member in a desired orientation relative to the housing.

### BRIEF DESCRIPTION OF THE DRAWINGS

These and other features and advantages of the connector assemblies and methods disclosed herein will be more fully understood by reference to the following detailed description in conjunction with the attached drawings in which like reference numerals refer to like elements through the different views. The drawings illustrate principles of the connector assemblies and methods disclosed herein and, although not to scale, show relative dimensions.

FIG. 1 is a exploded view of an exemplary embodiment of a connector assembly;

FIG. 2 is a side elevational view of the connector assembly of FIG. 1;

FIG. 3 is a top view of the connector assembly of FIG. 1;

FIG. 4 is a side elevational view in cross section of the connector assembly of FIG. 1 taken along the line A-A of FIG. 3, illustrating the a bone anchor and a fixation element positioned within the connector assembly;

FIG. 5 is a perspective view of the anchor receiving member of the connector assembly of FIG. 1;

FIG. 6 is a top view of the anchor receiving member of FIG. 5;

FIG. 7 is a side elevational view of the anchor receiving member of FIG. 5;

FIG. 8 is a perspective view of the housing of the connector assembly of FIG. 1;

FIG. 9 is a top view of the housing of FIG. 8;

FIG. 10 is a front view of the housing of FIG. 8;

FIG. 11 is a side view of the housing of FIG. 8;

FIG. 12 is a cross sectional view of the housing of FIG. 8 taken along the line B-B of FIG. 9;

FIG. 13 is a perspective view of the intermediate member of the connector assembly in FIG. 1;

FIG. 14 is a top view of the intermediate member of FIG. 13;

FIG. 15 is a side view of the intermediate member of FIG. 13; and

FIG. 16 is a cross sectional view of the intermediate member of FIG. 13 taken along the line C-C of FIG. 15.

### DETAILED DESCRIPTION

Certain exemplary embodiments will now be described to provide an overall understanding of the principles of the structure, function, manufacture, and use of the connector assemblies and methods disclosed herein. One or more examples of these embodiments are illustrated in the accompanying drawings. Those of ordinary skill in the art will understand that the connector assemblies and methods specifically described herein and illustrated in the accompanying drawings are non-limiting exemplary embodiments and that the scope of the present invention is defined solely by the claims. The features illustrated or described in connection with one exemplary embodiment may be combined with the features of other embodiments. Such modifications and variations are intended to be included within the scope of the present invention.

The articles “a” and “an” are used herein to refer to one or to more than one (i.e. to at least one) of the grammatical object of the article. By way of example, “an element” means one element or more than one element.

The terms “comprise,” “include,” and “have,” and the derivatives thereof, are used herein interchangeably as comprehensive, open-ended terms. For example, use of “comprising,” “including,” or “having” means that whatever element is comprised, had, or included, is not the only element encompassed by the subject of the clause that contains the verb.

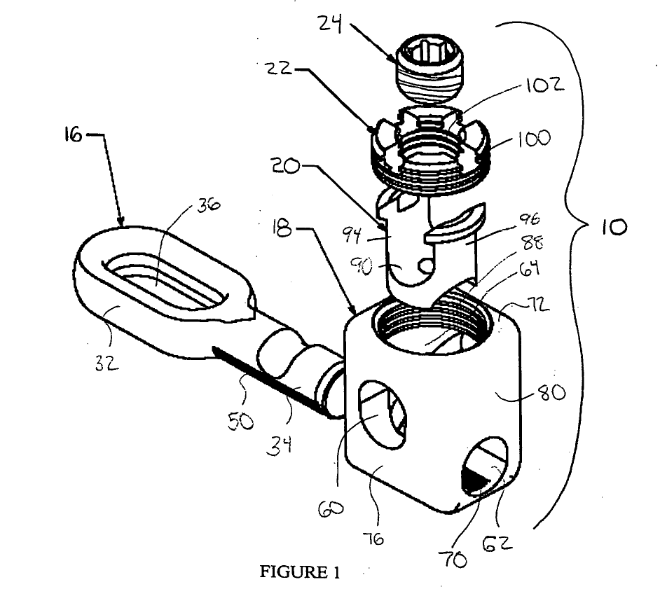

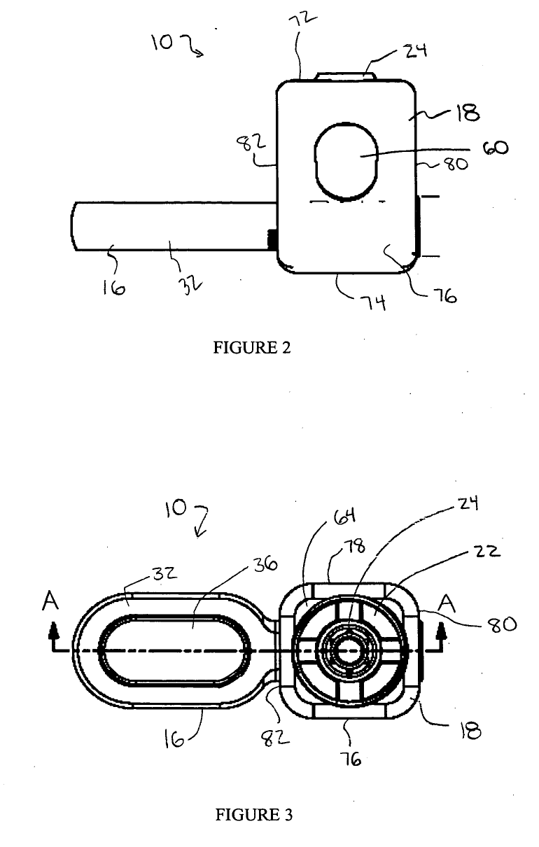

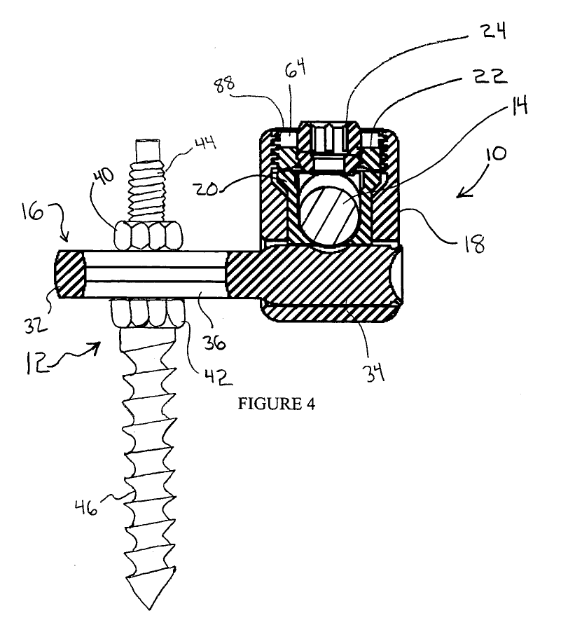

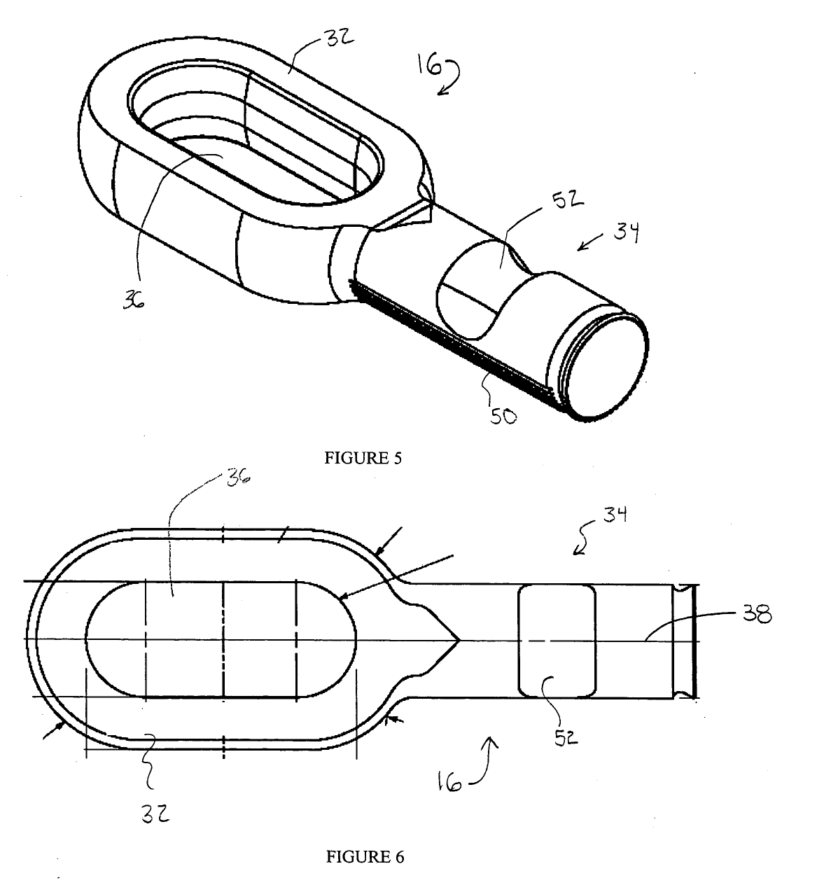

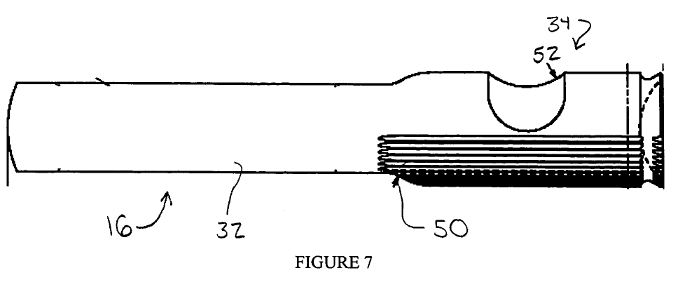

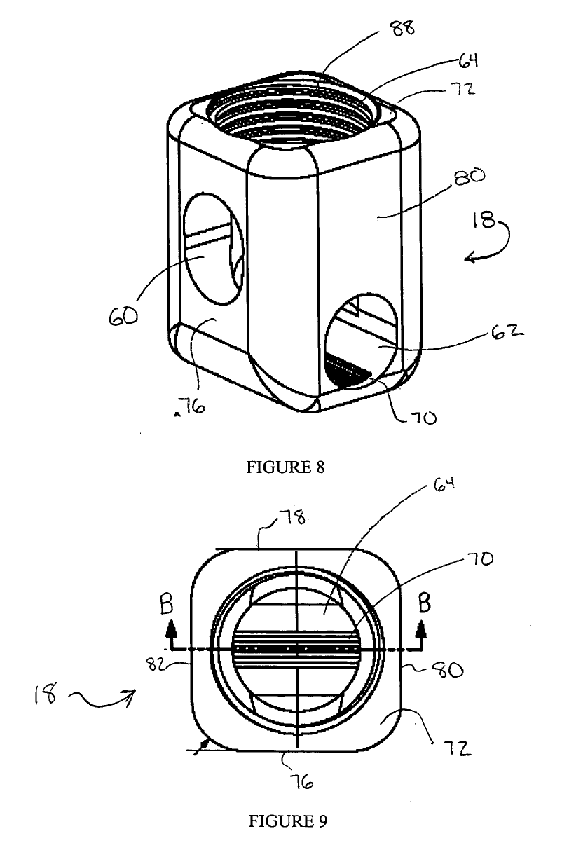

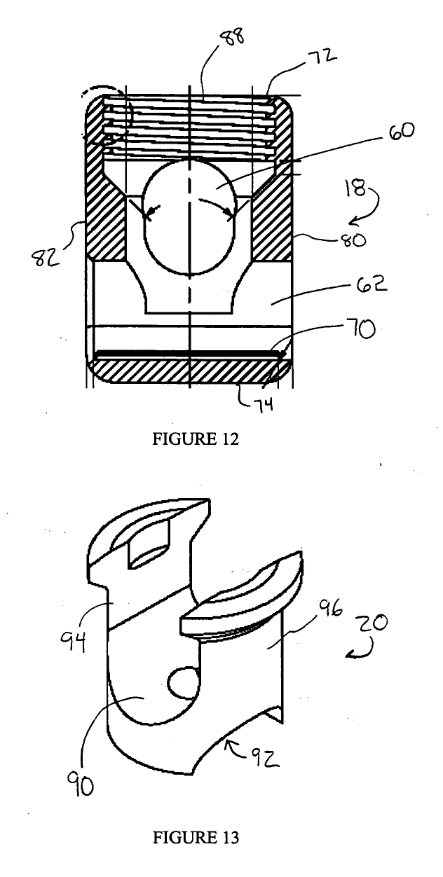

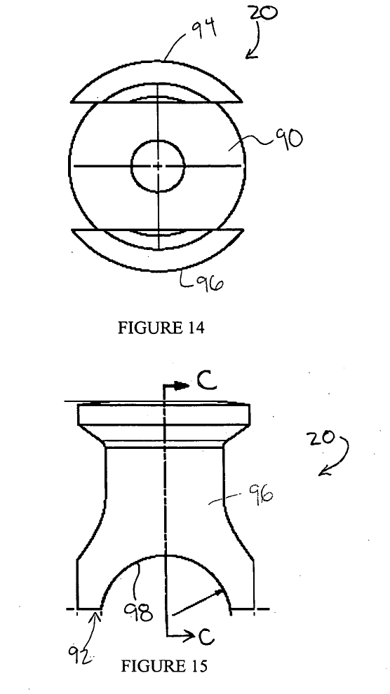

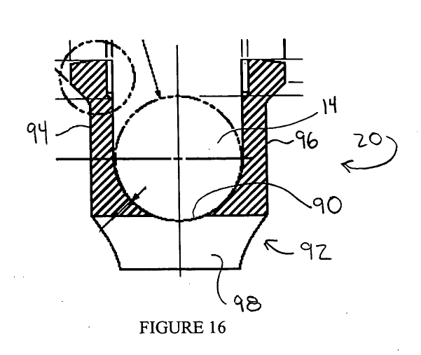

FIGS. 1-16 illustrate an exemplary embodiment of a connector assembly 10 for connecting a bone anchor 12, illustrated in FIG. 4, to a fixation element 14. The exemplary connector assembly 10 includes an anchor receiving member 16, a housing 18, an intermediate member 20 positionable within the housing 18 and receiving the fixation element, a first closure member 22 and a second closure member 24. The exemplary connector assembly 10 may be employed to connect a bone anchor, such as, for example, a bone screw, bolt, hook, or wire to a fixation element, such as a plate, rod, or cable. The connector assembly 10 may be used in orthopedic surgery to facilitate alignment and/or fixation of a desired relationship between anatomic structures and may be particularly suited to spinal surgery. For example, the exemplary connector assembly 10 may be employed to connect a bone anchor anchored to a vertebra to a spinal fixation element such as a spinal rod. In the exemplary embodiment, for example, the anchor receiving member 16 connects the bone anchor 12 to the housing 18, which receives the fixation element, for example, a rod 14, as illustrated in FIG. 4. The anchor receiving member 16, in the exemplary embodiment, may be rotated relative to the housing 18 to facilitate connection of the bone anchor to the fixation element in a wide variety of orientations. As described in more detail below, the exemplary connector assembly 10 permits the fixation element to be secured to the connector assembly 10 independent of securing the anchor receiving member 16 in a desired orientation relative to the housing 18 of the connector assembly 18.

The exemplary connector assembly 10 may be used with any type of bone anchor, including, for example, hooks, screws, such as monoaxial screws, bolts, and polyaxial screws, and wires. In the exemplary embodiment, for example, the connector assembly 10 is illustrated connecting a bone anchor 12 having distal bone engaging threads 46, proximal threads 44 for engaging a fastener, such as a nut 40, and an integral shoulder 42 positioned between the distal threads 46 and the proximal threads 44. Similar bone anchors and other exemplary bone anchors are described in U.S. Pat. Nos. 4,854,311; 4,648,388; 5,129,900; 5,312,404; 5,741,255; 5,743,907; and 6,080,156. Each of the following patents is incorporated herein by reference.

The exemplary connecting element may be used with any type of fixation element including, for example, rods, plates, tethers, and cables. In the exemplary embodiment, the connector assembly 10 is illustrated connecting a smooth rod 14 to a bone anchor 12.

Referring to FIGS. 1-7, the anchor receiving member 16 of the exemplary connector assembly 10 may include a first portion 32 configured to receive a bone anchor and a second portion 34 that is configured to be received by the housing 18. In the exemplary embodiment, the first portion 32 of the anchor receiving member 16 includes an opening 36 through which at least a portion of a bone anchor may be positioned. The size and shape of the opening 36 may be varied depending on, for example, the type of bone anchor and the surgical procedure being performed. In certain embodiments, for example, the opening 36 may be generally circular in shape. In other embodiments, including the exemplary illustrated embodiment, the opening 36 is generally elliptical in shape, e.g. the opening 36 is an elongated slot. The slot may have a central axis that is oriented parallel to or is coincident with a longitudinal axis 38 of the anchor receiving member 16, as illustrated in FIG. 6. The slot permits the bone anchor to be adjusted relative the anchor receiving member 16, for example, in a direction parallel to the longitudinal axis 38 of the anchor receiving member 16. The length of the slot in a direction parallel to the longitudinal axis may be varied depending on the amount of adjustment desired.

The bone anchor may be fixed to the first portion 32 of the anchor receiving member 16 by any conventional fastener. In the illustrated exemplary embodiment, an internally threaded nut 40 may be employed to fasten the bone anchor 12 to the anchor receiving member 16. The exemplary nut 40 engages the proximal threads 44 of the bone anchor 12. The nut 40 may be advanced distally to engage the proximal surface of the first portion 32 of the anchor receiving member 16 and may be tightened against the proximal surface to clamp the first portion 32 between the nut 40 and the shoulder 42 of the bone anchor 12.

The second portion 34 of the anchor receiving member 16 may be configured to be positioned within a passage of the housing 18 and to facilitate rotation of the anchor receiving member 16 about the longitudinal axis 38 and relative to the housing 18. In the exemplary embodiment, for example, the second portion 34 is generally cylindrical shape having, for example, a generally circular cross section. The second portion 34, in the exemplary embodiment, may be smooth or, as in the exemplary embodiment, may include one or more teeth 50 provided on the outer surface of the second portion. Meshing engagement of the teeth 50 with one or more teeth 70 provided on the passage of the housing 18 receiving the second portion 34 of the anchor engaging member 16 inhibits rotation of the anchor receiving member 16 about the longitudinal axis 38 and relative to the housing 18. Each of the teeth 50 may be aligned parallel to the longitudinal axis 38 of the anchor receiving member 16. Any number of teeth 50 may be provided. The teeth 50 may be provided about a portion of the second portion or the entire extent of the second portion. In the illustrated embodiment, a plurality of teeth 50 are provided on the distal surface of the second portion and have an arcuate extent of approximately 180° about the longitudinal axis 38 of the second portion.

The second portion 34 of the anchor receiving member 16 may include a groove 52 to facilitate engagement of the intermediate member 20 with the second portion 34 of the anchor receiving member 16 and minimize motion of the anchor receiving member 16 along the longitudinal axis 38 of the anchor receiving member 16. The groove 52 may have an arcuate extent of up to 360° about the longitudinal axis 38 of the second portion 34. For example, in the illustrated embodiment, the groove 52 has an arcuate extent of approximately 180° about the longitudinal axis 38 of the second portion. In other exemplary embodiments, a groove 52 may not be provided and the intermediate member 20 engages the outer surface of the second portion 34.

Referring to FIGS. 1-4 and 8-11, the housing 18 of the exemplary embodiment of the connector assembly 10 may include a first passage 60 for receiving a fixation element, a second passage 62 for receiving a portion of the anchor receiving member 16, such as the second portion 32, and a third passage 64 for receiving the intermediate member 20 and one or more closure members. The size and shape of the housing 18 may vary depending on, for example, the bone anchor and/or the fixation element employed. In the exemplary embodiment, the housing 18 is generally block-shaped, although one skilled in the art will appreciate that the housing 18 may have other shapes, including, for example, a cylindrical shape. The housing 18 of the exemplary connector assembly 10 has a top surface 72 opposed and parallel to a bottom surface 74, a first side surface 76 opposed and parallel to a second side surface 78, and a third side surface 80 opposed and parallel to a fourth side surface 82. The first, second, third and fourth side surfaces extend between the top surface 72 and the bottom surface 74.

In the exemplary connector assembly 10, the first passage 60 of the housing 18 extends between and is generally perpendicular to the first side surface 76 and the second side surface 78. The size and shape of the first passage 60 may be varied depending on, for example, the fixation element employed. In the case of a spinal rod, as in the illustrated embodiment, for example, the first passage 60 may have a generally oval cross section formed by a pair of circular openings having centers (C₁ & C₂, respectively), which are offset along a central axis 84, as illustrated in FIG. 11. The diameter of the circular openings which cooperate to form the first passage 60 may be selected to permit the fixation element to move between the upper and lower portions of the first passage 60. The pair of circular openings may have the same or different diameters. In the illustrated embodiment, for example, the diameter of the lower circular opening (C₂) is less than the diameter of the upper circular opening (C₁). The diameter of the lower circular opening (C2) may, in certain exemplary embodiments, be less than the diameter of the fixation element, e.g., the rod. In certain exemplary embodiments, the diameter of the lower circular opening (C2) may be selected to provide an interference fit when the fixation element, e.g., the rod, is seated in the rod seat of the intermediate member.

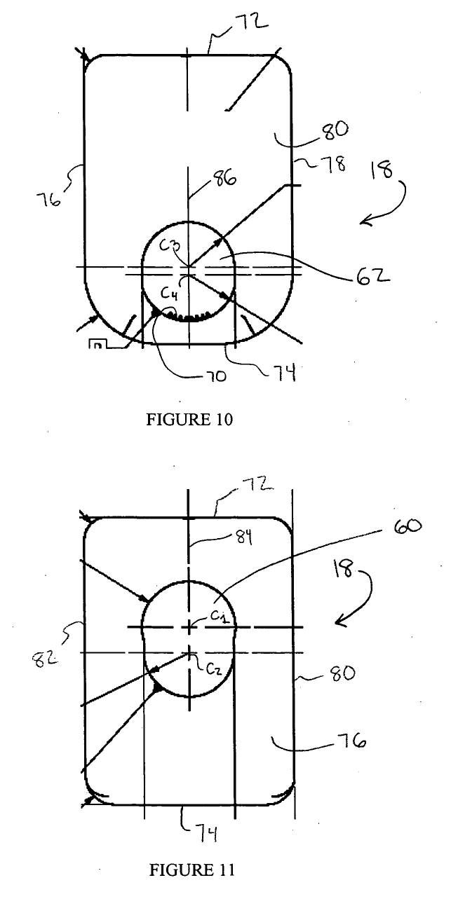

In the exemplary connector assembly 10, the second passage 62 of the housing 18 extends between and is generally perpendicular to the third side surface 80 and the fourth side surface 84. In the illustrated embodiment, the second passage 62 is oriented generally perpendicular to the first passage 60 and generally perpendicular to the third passage 64. The second passage 62, in the exemplary embodiment, is offset distally from the first passage 60 along the axis of the third passage 64. The size and shape of the second passage 62 may be varied depending on, for example, the size and shape of the second portion 34 of the anchor receiving member 16. In the case of a cylindrically shaped second portion 34, as in the illustrated embodiment, for example, the second passage 62 may have generally oval cross section formed by a pair of circular openings having centers (C₃ & C₄, respectively), which are offset along a central axis 86, as illustrated in FIG. 10. The diameter of the circular openings (C₃ & C₄) which cooperate to form the second passage 62 may be selected to permit the second portion 34 of the anchor receiving member 16 to rotate within the second passage 62 and move between the upper and lower portions of the second passage 62. The pair of circular openings may have the same or different diameters. As discussed above, the second passage 62 may include one or more teeth 70 for selective engagement with one or more teeth 50 provided on the second portion 34 of the anchor receiving member 16. In the illustrated embodiment, a plurality of teeth 70 are provided on the bottom inner surface of the second passage 62 and are oriented in a direction parallel to the axis of the second passage 62.

In the exemplary connector assembly 10, the third passage 64 of the housing 18 extends from the top surface 72 toward the bottom surface 74. The third passage 64 intersects and, in the exemplary embodiment, is generally perpendicular to the first passage 60 and the second passage 62. The size and shape of the third passage 64 may be varied depending on, for example, the size and shape of the intermediate member 20. In the case of an intermediate member 20 having a generally circular cross section, as in the illustrated embodiment, for example, the third passage 64 may be generally cylindrical in shape with a generally circular cross section. The proximal end of the third passage 64 may have internal threads or another engagement mechanism for engaging a closure member. In the illustrated embodiment, for example, the third passage 64 has internal threads 70 for engaging external threads provided on the first closure member.

Referring to FIGS. 1-4 and 13-16, the intermediate member 20 of the exemplary connector assembly 10 may be positionable within the third passage 64 of the housing 18. The intermediate member 20 may include a seat 90 for receiving the fixation element and may have a distal end 92 configured to engage a portion, e.g., the second portion 34, of the anchor receiving member 16. The size and shape of the intermediate member 20 may be varied depending on, for example, the fixation element and the housing employed. In the illustrated embodiment, for example, the intermediate member 20 has a pair of opposed legs 94, 96 that define a U-shaped slot for receiving the fixation element. In the exemplary embodiment, the base of the U-shaped slot defines an arcuate rod seat 90 having a diameter approximate to the diameter of the fixation element, e.g., the rod 14. In the exemplary embodiment, the distal end 92 of the intermediate member 20 may have an arcuate surface 98 having a curvature selected to facilitate engagement with the second portion 34 of the anchor receiving member 16. For example, in embodiments, such as the illustrated embodiment, employing an arcuate groove 52, the arcuate surface 98 may have a curvature selected to approximate the curvature of the arcuate groove 52.

Referring to FIGS. 1-4, the first closure member 22 of the exemplary connector assembly 10 may be positionable within the third passage 64 of the housing 18 to engage the intermediate member 20. In the exemplary connector assembly, the first closure member 22 may be positioned within the third passage to engage the proximal ends of the legs 94, 96 of the intermediate member 20. The first closure member 22 may be selectively advanced within the third passage 64 to force the distal end 92 of intermediate member 20 into engagement with the second portion 34 of the anchor receiving member 16 to inhibit rotation of the anchor receiving member 16. The first closure member 22 may have an engagement mechanism to permit engagement of the first closure member 22 with the housing 18. In the illustrated embodiment, for example, the first closure member 22 includes external threads 100 for engagement with internal threads 88 provided at the proximal end of the third passage 64. One skilled in the art will appreciate that engagement mechanisms other than threads may be employed. The size and shape of the first closure member 22 may be varied depending on, for example, the size and shape of the third passage 64 of the housing. In the illustrated embodiment, for example, the first closure member 22 is an annular set screw having external threads 100 and a threaded central opening 102 for receiving the second closure member 24.

The second closure member 24 of the exemplary connector assembly 10 may be positionable within the third passage way 64 to engage a fixation element positioned within the seat 90 of the intermediate member 20. For example, the second closure member 24 may be advanced through the first closure member 22 into the U-shaped slot of the intermediate member 20 to engage and secure a fixation element seated at the base of the U-shaped slot. The second closure member 24, in the illustrated embodiment, may be an inner set screw have external threads, or other engagement mechanism, for engaging the threaded central opening 102 of the first closure mechanism 24.

In use, the exemplary connector assembly 10 may be employed to connect a bone anchor with a fixation element by, for example, positioning a portion of a bone anchor within the opening 36 of the anchor receiving member 16 of the connector assembly 10 and by positioning a fixation element, e.g., a rod 14, within the first passage 60 of the housing 18 of the connector assembly 10. The anchor receiving member 16 may be rotated relative to housing 18 to adjust the orientation of the bone anchor relative to the fixation element. For example, the bone anchor 12 may be positioned within the opening 36 of the anchor receiving member 16 and the housing 18 may be rotated relative to the anchor receiving member 16 to facilitate positioning of the fixation element in the first passage 60 of the housing 18. Alternatively, the fixation element may be positioned within the first passage 60 of the housing 18 and the anchor receiving member 16 may be rotated to facilitate positioning of the bone anchor in the opening 36 of the anchor receiving member 16. Moreover, the fixation element and the bone anchor may be oriented relative to one another after both are coupled to the connector assembly by rotating the anchor receiving member 16 relative to the housing 18. Once the fixation element is positioned in first passage 60, the fixation element is free to move within the first passage 60, both along the axis of the first passage 60 and transverse to the axis of the passage (e.g., toward and away from the top surface 72 of the housing 18), until the fixation element is secured within the housing.

In the exemplary embodiment, the anchor receiving member 16 may be secured in a desired orientation relative to the housing independent of securing the fixation element relative to the housing 18. For example, the first closure member 22 may be advanced into contact with the proximal surface of the intermediate member 20 to force the intermediate member 20 into engagement with the second portion 34 of the anchor receiving member 16 and thereby inhibit relative rotation between the anchor receiving member 16 and the housing 18. In embodiments employing teeth 50 and teeth 70, advancement of the first closure member 22 into contact with intermediate member forces the teeth 50 and teeth 70 into meshing engagement to inhibit rotation. The second closure member 24 may be advanced independently of the first closure member 22 into engagement within the fixation element to secure the fixation element within the seat 90 of the intermediate member 20 and thereby inhibit relative motion between the fixation element and the connector assembly 10. For example, FIG. 4 illustrates the first closure member 22 engaged with the intermediate member 20 causing the intermediate member 20 to engage the second portion 34 of the anchor receiving member 16 to fix the anchor receiving member 16 against rotation and the second closure member 24 displaced from the fixation element, e.g., rod 14, such that the fixation element is adjustable relative to the housing 18 of the connector assembly 10.

While the connector assemblies and methods of the present invention have been particularly shown and described with reference to the exemplary embodiments thereof, those of ordinary skill in the art will understand that various changes may be made in the form and details herein without departing from the spirit and scope of the present invention. Those of ordinary skill in the art will recognize or be able to ascertain many equivalents to the exemplary embodiments described specifically herein by using no more than routine experimentation. Such equivalents are intended to be encompassed by the scope of the present invention.

1. A connector assembly for connecting a bone anchor to a fixation element, the connector assembly comprising:
an anchor receiving member having a first portion and a second portion, the first portion having an opening for receiving at least a portion of a bone anchor;
a housing having a first passage for receiving a fixation element, a second passage for receiving the second portion of the anchor receiving member, and a third passage intersecting the first passage and the second passage, the second passage being oriented at an angle to the first passage;
a intermediate member positionable within the third passage, the intermediate member having a seat for receiving the fixation element and a distal end configured to engage the second portion of the anchor receiving member;
a first closure member positionable within the third passage to engage the intermediate member; and
a second closure member positionable within the third passage to engage a fixation element positioned in the seat of the intermediate member.

2. The connector assembly of claim 1, wherein the second portion of the anchor receiving member is generally cylindrical in shape.

3. The connector assembly of claim 2, wherein the distal surface of the second portion includes a plurality of teeth for engagement with a plurality of teeth provided on the second passage of the housing.

4. The connector assembly of claim 2, wherein the second portion has a groove formed therein for engagement with the distal end of the intermediate member.

5. The connector assembly of claim 1, wherein the second passage is oriented perpendicular to the first passage and perpendicular to the third passage.

6. The connector assembly of claim 1, wherein a proximal end of the third passage includes threads for engaging threads provided on the first closure member.

7. The connector assembly of claim 1, wherein the first closure member is an outer set screw having external threads for engaging threads provided on a proximal end of the third passage.

8. The connector assembly of claim 7, wherein the outer set screw is annular in shape and includes a threaded central opening for receiving the second closure member.

9. The connector assembly of claim 8, wherein the second closure member is an inner set screw having external threads for engaging the threaded central opening of the outer set screw.

10. The connector assembly of claim 1, wherein the intermediate member has a pair of opposed legs defining a U-shaped slot for receiving the fixation element.

11. A connector assembly for connecting a bone anchor to a spinal rod, the connector assembly comprising:
an anchor receiving member having a first end portion and a cylindrically shaped second end portion, the first end portion having a slot for receiving at least a portion of a bone anchor;
a housing having a first passage for receiving a spinal rod, a second passage for receiving the second end portion of the anchor receiving member, and a third passage intersecting the first passage and the second passage, the second passage being oriented perpendicular to the first passage, the second end portion of the anchor receiving member being selectively rotatable about an axis of the second passage when positioned in the second passage;
a generally U-shaped member positionable within the third passage, the generally U-shaped member having a pair of spaced apart legs defining a rod slot for receiving the spinal rod, the generally U-shaped member having a distal end having an arcuate surface for engaging the cylindrically shaped second portion of the anchor receiving member;
a first closure member positionable within the third passage to engage the generally U-shaped member; and
a second closure member positionable within the first closure member and within the rod slot to engage a spinal rod positioned within the rod slot of the generally U-shaped member,
wherein advancement of the first closure member into engagement with the generally U-shaped member forces the arcuate surface of generally U-shaped member into engagement with the second end portion of the anchor receiving member to thereby inhibit rotation of the second end portion about the axis of the second passage and wherein engagement of the second closure member with the spinal rod positioned in the rod slot inhibits motion of the spinal rod relative to the housing.

12. A method of connecting a bone anchor with a fixation element, the method comprising:
positioning a portion of a bone anchor within an opening of an anchor receiving member of a connector assembly;
positioning a fixation element within a passage of a housing of the connector assembly;
rotating the anchor receiving member relative to a housing;
securing the anchor receiving member in a desired orientation relative to the housing; and
securing the fixation element relative the housing independently of securing the anchor receiving member in a desired orientation relative to the housing.

13. The method of claim 12, further comprising securing the bone anchor to the anchor receiving member.

14. The method of claim 12, wherein securing the anchor receiving member in a desired orientation relative to the housing comprising advancing a first closure member in the housing.

15. The method of claim 14, wherein the first closure member is advanced into engagement with an intermediate member positioned within the housing of connector assembly between a portion of the anchor receiving member and the first closure member.

16. The method of claim 12, wherein securing the fixation element relative the housing comprises advancing a second closure member into engagement within the fixation element.
